(19) United States
(12) Patent Application Publication
(10) Pub. No.: US 2007/0233174 A1
(43) Pub. Date: Oct. 4, 2007

(54) Trapping Filter for Blood Vessel

(76) Inventors: Gordon Hocking, Tokyo (JP); Cheng Zheng Hui, Shenzhen (CN)

Correspondence Address: ROBERT C. KAIN, JR., 750 SOUTHEAST THIRD AVENUE, SUITE 100, FT LAUDERDALE, FL, 333161153, US

(21) Appl. No.: 11/554,055

(22) Filed: Oct. 30, 2006

(30) Foreign Application Priority Data
Apr. 1, 2006 (JP) 2005-133051

(51) Int. Cl. A61M 29/00

(52) U.S. Cl. 606/200

(57) ABSTRACT

The present invention provides the filter from the wire elements only, which certainly traps the thrombus and others, and easily visualizes from extra-corporeal and trapping system. The solution set forth in the present invention involves an expandable trapping filter, which is consist of plural knitted metallic wires with elastic or shape memory nature. The filter has two bundled parts in the distal and proximal ends of plural wires. When this filter expands at the intended tubular cavity of body part, each wire flips over to form parasol shape moving proximal bundled to distal bundled to catch a thrombus and others. Trapping system is deliverable from the distal edge of sheath which is interiorly loaded and is able to move in and out by the shaft movement along the penetrating shaft with the of proximal bundle immobilization.

The present invention provides an expandable trapping filter, which consists of plural knitted metallic wires with elastic or shape memory nature, for a blood vessel filter.

### TECHNICAL BACKGROUND

For the stenosis at the coronary artery, carotid artery and venous vessel grafts, the therapy for blood flow securement is being done by catheter application with stenting and/or balloon dilatation. Even though the treatment attains the blood flow, the releasing of the plaque or thrombus during the treatment, downward blood flow from the target in the blood vessel sometimes causes the no-reflow. In such a case, recirculation is not attainable. The resulting side effect sometimes causes a severe condition.

In light of this background, many trapping systems for thrombus have been proposed. (Refer to Patent 1, Patent 2 and Patent 3). Conventional thrombus trapping system is composed of knitted component 70 with plural spiral wires 71. Component is the barrel shape with middle expanded figure by distal bundle 72 and proximal bundle 73, the filter part is located at the center to distal end. Mesh of filter 74 is 50 micro to 1000 micro in the range of center to distal end. For the bulky thrombus, it is possible to catch the thrombus with plural wire knit or without knitted mesh. Quantity of filter wire is favorable to be 8-16. (Patent 1)
Patent-1—2004-97807
Patent-2—2001-514554
Patent-3—2002-505151

### DISCLOSURE OF INVENTION

### PROBLEMS SOLVED BY THIS INVENTION

The filter created by the knitted crossing of wires at the distal side is easier to insert and extend from a sheath compared to foldable material and/or more plastic sheet filter. At the insertion, filter material tends to wrinkle; there is also a fault when the filter is expanded by wire. Moreover, if filter itself is foldable, the mesh pore size is not constant.

There is another idea of using plural metallic wires. However, conventional metallic filter has a risk to filtrate the trapped thrombus as the filter is retrieved into sheath. Also, the filter with only 16 wires does not have trapping function.

Furthermore, metallic wire is generally applied by Nickel-Titanium metallic alloy, which has poor radiopaque characteristics. This is a problem in that the filter is not visible by physicians.

This invention aims to provide the trapping system with filter which is composed of only wire for reliable trapping of thrombus and other items, and is visible from extraccoporial positions.

### SUMMARY OF THE INVENTION

The present invention provides the filter from the wire elements only, which certainly traps the thrombus and others, and easily visualizes from extra-corporeal and trapping system. The solution set forth in the present invention involves an expandable trapping filter, which is consist of plural knitted metallic wires with elastic or shape memory nature. The filter has two bundled parts in the distal and proximal ends of plural wires. When this filter expands at the intended tubular cavity of body part, each wire flips over to form parasol shape moving proximal bundled to distal bundled to catch a thrombus and others. Trapping system is deliverable from the distal edge of sheath which is interiorly loaded and is able to move in and out by the shaft movement along the penetrating shaft with the of proximal bundle immobilization.

### BRIEF EXPLANATION OF THE DRAWINGS

FIG. 1 shows the first example of the form which carries out this invention, and is a sectional view which a trapping filter contracts and is stored in the sheath of trapping equipment.

FIG. 2 is a sectional view which the trapping filter which is the first example of a form which carries out this invention, and was moved out of the sheath of trapping equipment is extending within a blood vessel.

FIG. 3 is an elevational view seen from the tip side of the trapping filter which FIG. 2 extended.

FIG. 4 is a back figure seen from the proximal side of the trapping filter which FIG. 2 extended.

FIG. 5 is an outline figure of the tip portion of the trapping filter which FIG. 2 extended.

FIG. 6 is the 2nd example of the form which carries out this invention, and is a sectional view by which the trapping filter is contracted and stored in the sheath of trapping equipment.

FIG. 7 is a sectional view which the trapping filter which is the 2nd example of the form which carries out this invention, and was moved out of the sheath of trapping capture equipment is extending within a blood vessel.

FIG. 8 is a back figure seen from the proximal side of the trapping filter which FIG. 7 extended.

FIG. 9 is a side view which shows another mode in the case of attaching a trapping filter in the shaft tip part of the trapping equipment in the 2nd example of the form which this invention carries out.

FIG. 10 is a sectional view which met the I-I line of the base-edge union part of FIG. 9.

FIG. 11 is an expansion perspective diagram of the proximal union part of FIG. 9.

FIG. 12 is an outline figure of the conventional trapping filter

### EXPLANATION OF ITEMS IN DRAWINGS

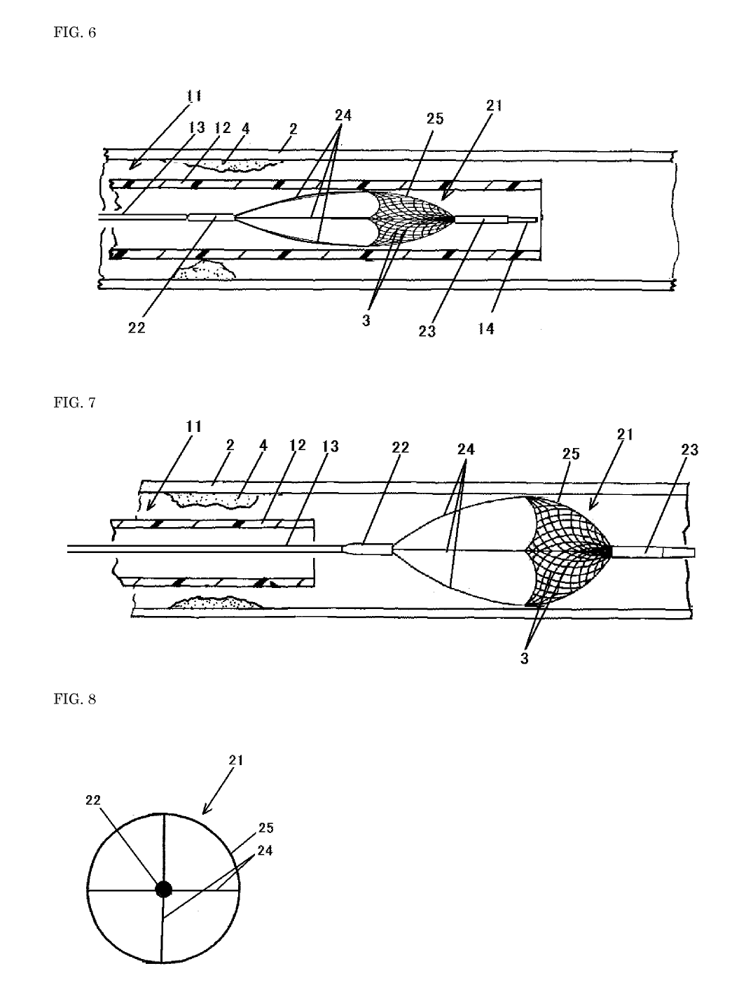

The present invention provides an expandable trapping filter, which is consist of plural knitted metallic wires with elastic or shape memory nature as a blood vessel filter. In the drawings, the following numerals identify the following items: 1. Trapping filter in general; 2. Blood Vessel; 3. Guide Wire in general; 4. Portion of Blood Vessel narrowed due to stenosis (stenosised portion); 5. Proximal Tip binding point material for filter; 6. Distal binding point material for filter; 7. Bending and turning portion of filter wire; 11 no. Trapping equipment; 11. Sheath; 13. Guide Wire Shaft; 21. Trapping filter (FIG. 6); 22. Proximal binding point material for filter (FIGS. 6, 7); 23. Distal Tip binding point material for filter; 24. Non-filter portion; 25. Filter portion.

This invention is finalized using the plural elastic or shape memory metallic wire knitted or wire knitting permitting expansion. At the filter expansion, this makes reliable thrombus trap by expanding at the proximal bundle by strings for parasol shape with each wire flipping-over or parachute shape.

With the use of a partially radiopaque metal for the knitted filter wire, especially an impregnated core by radiopaque metal with exterior layer of elastic or shape memory metal wire, the filter of this invention can be visualized by medical equipment when the filter is in a blood vessel without the filter being in an expanding function.

The following describe characteristics of the composition and structure of trapping filter and trapping system of this invention.
(1) This invention is an expandable trapping filters, which is consist of plural knitted metallic wires with elastic or shape memory nature. The filter has two bundled parts in the distal and proximal ends of plural wires. When this filter expands at the intended tubular cavity of body part, each wire flips over to form parasol shape moving proximal bundled to distal bundled to catch a thrombus and others.
(2) This invention is expandable trappings filter, which is consist of plural knitted metallic wires with elastic or shape memory nature. The filter has two bundled parts in the dista and proximal ends of one to 12 wires. When this filter expands at the intended tubular cavity of body part, each wire flops over to form parasol shape moving proximal bundled part to distal bundled part to catch a thrombus and others.
(3) The trapping filter is a bundle of wires, without a mesh filter, having one to 12 wires from proximal bundled to bent over portion.
(4) The wire quantity is more than 20 in a preferred embodiment.

Using the thrombus trapping filter of this invention, such a flexible filter cloth etc. is unnecessary. Since the filter from a metallic, elastic and shape memory wire, this is able to put into sheath and expand in the blood vessel when takes out from the inside of a sheath, correctly. In this case, since each wire flips over and expands, the wire portion from the proximal bundled to bent-over portion, carries out the action, which makes a filter expand, respectively. For this reason, a filter turns to expand certainly in a blood vessel. Moreover, a filter part from a proximal bundled to flip-over portion and the bent-over portion to distal bundled, turns into a double structured filter and catches a thrombus certainly. On the other hand, the residuum tends to locate the near center or bundles area; the captured thrombus does not squeeze off from the filter mesh at the sheath tip wall when filter is recovered by the Sheath. Moreover, when a filter expands to a parachute shape, a captured thrombus is certainly holded at the pocket of the parachute shape. Especially the filter from 20 or more wires does not pass off the thrombus. Also when a filter is recovered in the sheath, the captured thrombus is not squeezed off by the sheath wall.

Moreover, the wires are the rebounding elasticity or shape memory nature metal in general, for example, metallic alloys of Nickel-Titanium, Copper-Zinc-Manganese and Iron-Manganese-Silicon. However, since some metallic alloys of Nickel-Titanium etc. are poor on X-rays radio pacifier, the observation of filter is not easy with such metals only. For this reason, to improve observation from the outside of the body by an X-rays radio pacifying metal (superior radiopacity, imaging nature), such as gold, platinum, palladium, tungsten, etc. are woven or coated or metallically blended or interior cored wires. Especially by the elastic or shape memory metal coverage of the radiopaque core, exterior metal maintains the mechanical strength and shape memory nature and forces the filter firm expansion without the drop or destroy of radiopaque metal. When the radiopaque metal is applied to the trapping system on the distal part of a shaft, both the bundle part of a filter, a guiding tip, etc., a motion of trapping equipment and a filter is correctly observable from the outside.

The form of implementation of this invention is hereafter explained with reference to an attachment drawing.

In addition, as for the trapping filter shape to trap the thrombus in accordance with the principles of the present invention, it is not necessarily restricted to the shape shown herein.

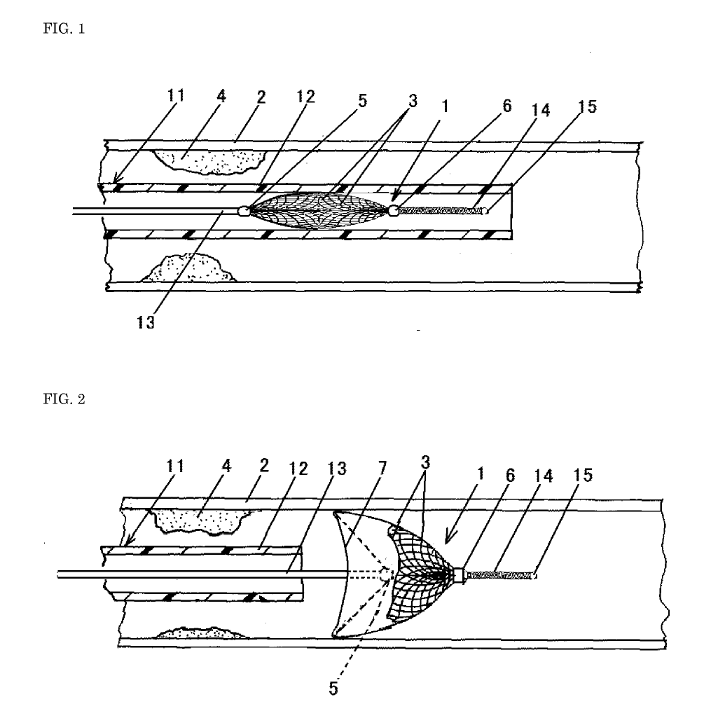

FIG. 1 is the 1st example of the form which carries out this invention, and is a sectional view wherein the trapping filter is contracted and stored in the sheath of trapping equipment.

FIG. 2 is the 1st example of the form which carries out this invention, and is a sectional view wherein the capture filter has been moved out of the sheath of trapping equipment and is expanding within a blood vessel.

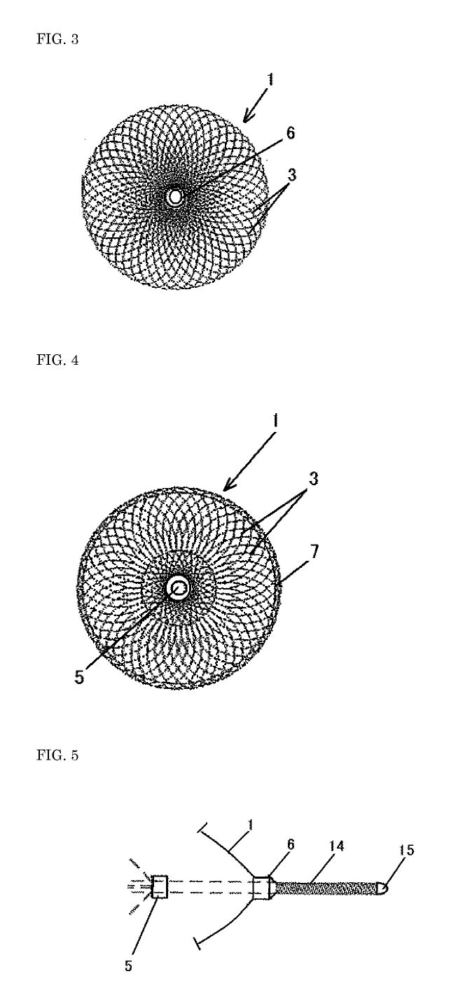

FIG. 3 is a distal view from the tip side of the trapping filter of FIG. 2 showing an expanded filter.

FIG. 4 is a proximal view from the proximal and of the trapping filter of FIG. 2.

FIG. 5 is an outline figure of the tip portion of the trapping filter of FIG. 2.

FIG. 6 is the 2nd example of the filter which carries out this invention, and is a sectional view of the trapping filter wherein the filter is contracted and stored in the sheath of trapping equipment.

FIG. 7 is a sectional view of the trapping filter per the 2^{nd} example wherein the filter was moved out or slid from the sheath of trapping equipment and is extending within a blood vessel.

FIG. 8 is a back view from the proximal end of the trapping filter of FIG. 7.

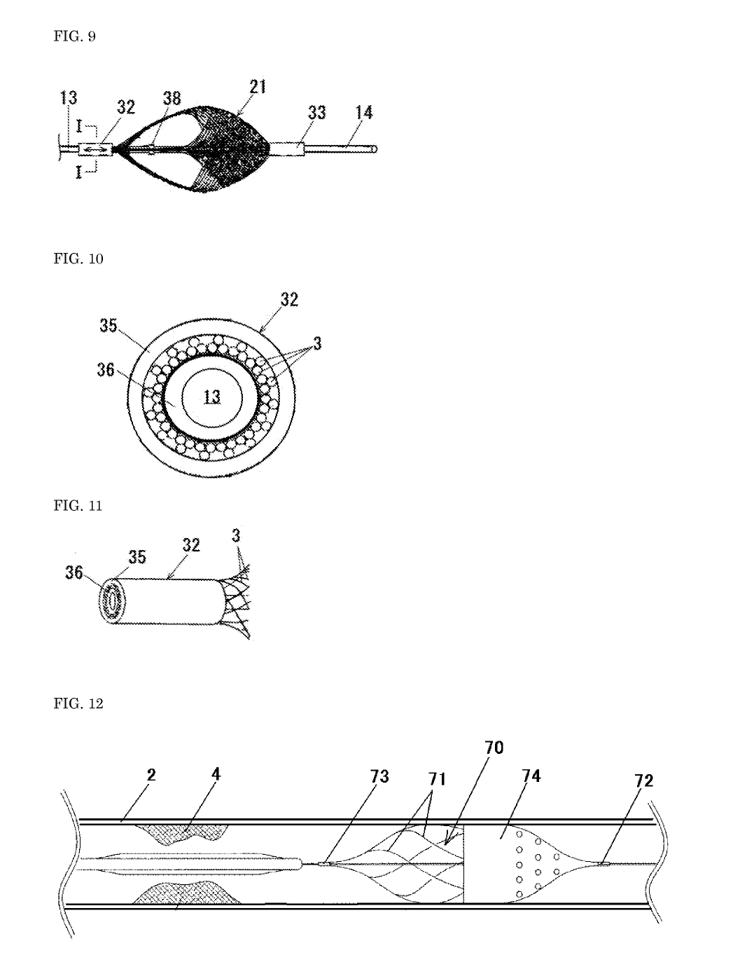

FIG. 9 is a side view of another mode in the case of attaching a trapping filter in the shaft tip part of the trapping equipment in the 2nd example of the invention.

FIG. 10 is a sectional view from alongside of the I-I line of the proximal bundled part of FIG. 9.

FIG. 11 is an expansion perspective drawing of the proximal bundle of FIG. 9.

As shown in FIGS. 1 and 2, the trapping filter 1 consists of plural knitted metallic wires 3. This filter expands at the intended tubular cavity 2 of body part, to catch thrombus 4. The trapping filter 1 has two bundled parts 5,6 in the distal and proximal ends of plural wires 3 which wires have an elastic or shape memory nature. At the time when filter 1 expands, each wire flips over, shown in FIGS. 2 and 4, to form parasol shape moving proximal wire bundle 5 to distal wire bundle 6 to catch a thrombus and other material in the blood vessel or body element. Especially, it is preferred that the proximal bundled wire portion 5 is positioned closer to distal bundled wire part 6 than the flip-over wire portion 7 of the wire filter.

In addition, the trapping device 11 to which the trapping filter 1 applies has the sheath 12 and the shaft (or guide wire) 13 that penetrates into the sheath 12. The shaft 13 can move in the sheath 12 in the direction of its axis. The distal binding part 5 of the trapping filter 1 is fixed to the tip of the shaft 13 and the trapping filter 1 is delivered out from the tip of the sheath 12 by movement of the sheath 12 or the shaft 13.

The tip binding part 6 of the trapping filter 1 is equipped with the coil-shaped guide chip 14 and the chip 14 has the top member 15.

Note that, in the examples of FIG. 1 and FIG. 2, the tip binding part 6 is not mounted on the tip of the shaft 13. Therefore, the tip binding part 6 need not necessarily to connect to the shaft 13 and the shaft 13 need not to exist between the distal binding part 5 and the tip binding part 6. This is the feature of this trapping filter 1 and saves spaces of members. However, the tip binding part 6 can be mounted on the tip of the shaft 6 so that it can slide through the shaft. Even in this case, the filter 1 can expand in the parasol shape when it moves out of the sheath 12.

As shown in FIG. 3 and FIG. 4, the trapping filter 1 can expand in the middle part that is knitted up with multiple metallic wires 3. The metal of the wire 3 has anti-elasticity, super-elasticity, and shape memory nature. Concrete metals for the metallic wire include Ni—Ti alloy, Cu—Zn—Mn alloy, Cu—Zn—Al alloy, Cu—Al—Ni alloy, and Fe—Mn—Si alloy. In particular, Ni—Ti alloy is desirable.

Metals and alloys above usually have ideal anti-elasticity and shape memory nature but they have lack of X-ray photography nature. It is difficult to observe it out of a human body using X-rays during an operation. Therefore, it is desirable that, in addition to metal above, metal with X-ray photography nature or opaque metal (so-called radiopaque metal) is included partially in the trapping filter 1 so that the trapping filter 1 can be observed out of a human body. Concrete radiopaque metal includes gold (Au), platinum (Pt), platinum/iridium (Pt/Ir), and tungsten (W) and in particular, AU, Pt, or Pt/Ir is desirable.

In addition, if the radiopaque metal is partially included, Pt wire, W wire, Au wire, Pt/Ir wire can be knitted into the Ni—Ti alloy wire above. Alternatively, the surface of the Ni—Ti alloy wire above may be coated with Pt, Pt/Ir, or Au, or Pt, Pt/Ir, or Au is used as a core with an outer layer of Ni—Ti alloy. In addition, Ni—Ti—Pt alloy or Ni—Ti—Au alloy may be partially included in the wire.

Particularly, it is desirable that the wire, for which the core of Au or Pt is wrapped or coated with Ni—Ti alloy, is used for a part or whole of the filter 1. If radiopaque metal is used for the core and a metallic wire, which is made of metal having anti-elasticity or shape memory nature, is used for its outer layer, the radiopaque metal never be observed during operation because of lack or damage. In addition, the outer-layered metal will surely expand the filter since it properly shows mechanical strength and shape memory nature. In this case, it is desirable that the core diameter of the radiopaque metal is in the range of 10 to 80% of the wire, especially 30 to 50%. If the diameter is less than the range above, it cannot be observed sufficiently. If the diameter exceeds the range above, anti-elasticity or shape memory nature will degrade.

The outer diameter of the wire is not especially restricted. It can be selected properly depending on thickness of the filter 1 when expanding and the size of trapped thrombus. The outer diameter of the wire usually ranges from 0.001 inch to 0.1 inch and the range from 0.001 to 0.02 inch is more desirable. If the wire diameter is within the range above, the filter 1 can be easily accommodated in the sheath 12 and it can exactly expand to a desired shape in a tube 2. Furthermore, each wire 3 may be knitted in any ever known knitting manner such as plain stitch and the knitting manner is not especially restricted as long as the mesh can be shaped for the filter 1. The mesh diameter (hole diameter) of the filter 1, which is formed with wires when being expanded, need to be within the range from 0.5 to 0.05 mm and if it is within the range, a thrombus or plaque can be trapped sufficiently.

In addition, it is desirable that the ratio of the reduced filter 1 itself to the expanded filter in the sheath is within the range from 1/10 to ½, especially ⅛ to ¼. If it is in such range, the outer diameter of the sheath 12 can be reduced as much as possible.

The number of wires 3 consisting of the trapping filter 1 need to be 20 or more. If the number of the metal wires 3 is 20 or more, sufficient elastic force can operate and the trapping filter 1 can expand accurately. In addition, the trapping filter 1 can accurately configure the mesh diameter above mentioned when expanding and it can surely trap a thrombus. Therefore, the number of the metal wires 3 for the trapping filter 1 need to range from 20 to 200, especially from 32 to 98. If the number of the metal wires 2 is less than 20, the trapping function of the filter 1 will degrade. If the number of the metal wires 3 exceeds 200, difficulty occurs on the filter configuration.

Note that the outer diameter of the trapping filter 1 when expanded is not restricted. It is necessary to select a different diameter depending on a blood vessel diameter of a diseased part.

In the trapping filter 1, each tip of multiple wires 3 is tied with each distal through ring-shaped binding members 5 and 6. The material of the binding members 5 and 6 may be metal or resin and ordinary SUS can be used for metallic material as well as metal above mentioned. The resin can include living-body adaptable resin such as polyurethane, polyethylene, polyester, polypropylene, polyamide, polytetrafluoroethylene, and polyvinylidene fluoride.

In addition, it is desirable that radiopaque metal such as gold (Au), platinum (Pt), platinum/iridium (Pt/Ir), and tungsten (W) is used partially or entirely for such binding members 5 and 6.

As shown in FIG. 4, a folded part 7 generates in each wire when expanded. It is desirable that the distal binding part 5 relatively comes close to the tip binding part 6 and in particular, the distal binding part 5 is located at the place more close to the tip binding part 6 than to the folded part 7 of the wire. In this case, since thrombosis accumulate in the vicinity of the distal binding part 5 and the sheath 12 has a given inner diameter, thrombosis can be smoothly collected without being filtered out by the edge of the sheath 12 when the filter size reduces.

The trapping filter 1 above mentioned is mounted on the tip part of the shaft 13 for the trapping device 11. The distal binding part 5 is fixed to the shaft 13 but the tip binding part 6 is not fixed to the shaft 13. The feature of the trapping filter 1, this invention, is that the filter can be expanded when the tip binding part 6 is mounted on the shaft 13 and when it is mounted so that it can slide. Alternatively, it is possible for the distal binding part 5 to slide along the shaft 13 and for the tip binding part 6 to be fixed to the shaft 13. In the examples of FIG. 1 and FIG. 2, since there is no shaft 13 between the distal binding part 5 and the tip binding part 6, the space of members can be saved.

The material of the shaft 13 includes Ni—Ti alloy, SUS, and other metal and the diameter of the shaft 13 is not limited especially (the example above uses 0.014 inch). The outer diameter on the tip part of the shaft 13 in vicinity of the distal binding part 5 tapers off gradually to the distal binding part. It is desirable that the diameter tapers as small as 0.012 to 0.1 inch. In addition, it is desirable that radiopaque metal is partially used for the shaft 13.

The shaft 13 penetrates into the sheath 12 at the distal end (not shown in Figure) of the trapping device and can move in the direction of sheath 12 axis. Therefore, the trapping filter 1 is delivered out of the tip end of the sheath 12 by relative movement of the shaft 13 at the distal end.

It is desirable that the sheath 12 consists of living-body adaptable synthetic resin such as polyurethane, polyethylene, polyester, polypropylene, polyamide, polytetrafluoroethylene, and polyvinylidene fluoride. The inner diameter of the sheath 12 need not be restricted especially and determined depending on the applied blood vessel and a size of the trapping filter 1. As shown in FIG. 5, the tip binding part 6 of the trapping filter 1 is equipped with the coil-shaped reversible guide chip 14 and the tip end of the chip 14 is equipped with the top member 15. The guide chip 14 may be ribbon-like shaped and its length usually ranges from 10 to 70 mm depending on a used blood vessel and a size of the trapping filter. The guide chip 14 is made of SUS and, as mentioned above, radiopaque metal or Pt may be included partially or entirely. Note that if the guide chip is ribbon-like shaped, it is desirable to be made of Pt. Furthermore, the radopaque metal above mentioned is desired to be used for the top member 15. It is desirable that the coil-shaped guide chip tapers with being thickened toward the end.

To use the trapping filter 1 and trapping device 11 of such structures, the trapping device 11 as shown in FIG. 1 is first retained near the target blood vessel. Then, the tip of the sheath 12 in which the trapping filter 1 is stored in s compressed state is inserted up to the peripheral side of angiostenosis site 4. Pulling the sheath 1 toward the distal side with the shaft 12 fixed will bring the trapping filter 1 out of the sheath 12 and make it expand, adhering to the vascular wall.

A prescribed surgery is conducted to capture thrombi from the angiostenosis site 4 and others, during which the trapping filter 1 can be correctly observed from outside. After the surgery, pushing the sheath 12 to the tip with the shaft 13 fixed will make the trapping filter 1 stored in the sheath 12 again. Finally, removing the thrombus trapping devise together will complete the percutaneous angioplasty.

In this practice, the trapping filter 1, when expanded, provides a parasol-type filter, eliminating the need for supplementary components such as flexible filter cloth. The trapping filter 1 is appropriately compressed and stored in the sheath 12 of the trapping device 11, which will expand without fail in blood vessel when taken out of the sheath 12, ensuring the capture of thrombus and debris. As each of wires 3 expands in a folded-back form, each of the wires from distal binding part 5 to folded-back part 7 works in a way to ensure expansion of filter 1. In addition, the filter 1 becomes a double filter from the folded-back part 7 to the tip binding part 6, ensuring capture of thrombi At the same time, when the filter 1 is stored in the sheath 12, thrombi and debris are collected to the center of the filter 1, in other words, near the binding part 5, thus less worry about drop of thrombus at the tip wall of the sheath 12 compared with the conventional way.

FIG. 6 indicates the 2nd example of the method to execute this invention and is the cross sectional view showing that the folded trapping filter is placed in the sheath of the trapping device. FIG. 7 also indicates the 2nd example of the method to execute this invention and is the cross sectional view showing that, after the said filter is moved out of the sheath of the trapping device, it expands in a blood vessel.

The trapping filter 21 in the 2nd example is different from Trapping filter 1 in the 1st example because the former has the following structure. This document does not include the details about how Trapping filter 21 in the 2nd example is similar with Trapping filter 1 in the 1st example.

As shown in FIG. 6 and FIG. 7, the trapping filter 21 that is made of expandable filter woven with multiple metallic wires 3 having anti-elasticity or shape memory nature expands in the lumen to capture the thrombus 4. The tip and distal parts of each of the above wires are bound. The metallic wires are woven to make a parachute shape with the distal side represented in non-filter part 22 and the tip side in filter part 23.

Detailed explanation of the trapping device 11 to which the trapping 21 is applied is omitted here as it is similar to the example No. 1. In the example of FIG. 6 and FIG. 7, the tip binding part 6 is attached to the shaft 13. The binding part 6 slides on it.

As described in FIGS. 3 and 4 for Trapping filter 1, several Wires 3 are used as material to weave Trapping filter 21. More specifically, Wire 3 is made from the elastic, super elastic or shape memory metal such as Ni—Ti alloy, Cu—Zn—Mn alloy, Cu—Zn—Al alloy, Cu—Al—Ni alloy and Fe—Mn—Si alloy. In addition to these alloys, the radiopaque metal is partially involved in the wire. This allows you to observe Trapping filter 1 from the outside of a human body. The external diameter of a wire of trapping filter 21, mesh, the number of wires, expansion multiples and so on are almost the same as those of Trapping filter 1. Additionally, the materials of the bonding members of Trapping filter 21, and the structures of Sheath 12, Shaft 13, Guidance chip 14 and Top member 15 of the trapping device are also almost the same as those of Trapping filter 1.

Trapping filter 21 is different from Trapping filter 1 in terms of the following items.

As shown in FIG. 1, the Trapping system 11 is placed at the beforehand near blood vessel aiming at thrombus at the use of the Trapping filter 21 and the trapping equipment. Then, the tip part of the Sheath 12 with the stored Trapping filter 1 is shifted to the distal side of the blood vessel Stenosis part 4. When a Sheath 12 is pulled to aproximal side (physician side) where a Shaft 13 is fixed, the Trapping filter 21 comes out from the inside of a Sheath 12 and expands and sticks to a blood vessel wall. After a predetermined operation, Sheath 12 is sled forward on holding a Shaft 13, a Filter 21 is again contained into a Sheath 12. When thrombus-trapping equipment is finally taken out wholly, a percutaneous blood-vessel-plasty is completed. In such a series of operations, the thrombus from the Stenosis part 4 etc. is captured in the Filter part 25 by the side of the tip Bundled component 23 side, and even when a Filter 21 is folded and recovered in a Sheath 12, a thrombus etc. is in the state where it was held firmly, and is caught firmly.

FIG. 9 is a side view showing another mode of holding a trapping filter in the shaft tip part of the trapping equipment in the 2nd example of this invention.

FIG. 9 shows another mode at the time of holding or attaching the trapping filter 21 in the shaft 13 of trapping equipment. That is, in the trapping equipment 11 which has a trapping equipment sheath and the shaft 13 loaded in this sheath, it is held possible [movement] at the shaft 13 to the length which the mobile proximal bundled part 32 of the capture filter 21 was attached in the tip part 13 of a shaft 13, and the proximal bundled part 32 defined in the direction of an axis (the direction of the arrow of FIG. 9) beforehand in the tip part of a shaft 13.

As shown in FIGS. 10 and 11, the proximal bundled part 32 consists of a coating ring component 35 and an inner ring component 36. The tip part of a wire 3 was inserted between the coating ring component 35 and the inner ring component 36, and has bundled together. The tip part of a shaft 13 is relatively inserted possible to slide in the inner ring component 36. Moreover, the tip bundled part 33 is being fixed to the tip part of a shaft 13. In addition, it does not necessarily need to be fixed and the tip bundled part 33 may also be held possible to slide at the shaft.

As shown in FIG. 9, it is the shaft tip part 13, and between the proximal bundled part 32 and the tip bundled part 33, in order to regulate the union part 32 and/or the slide move distance in the shaft of 33, the stopper ring 38 which serves as a diameter of a large from the diameter of inner of the above-mentioned inner ring component 36 fixes to a shaft 13, and is attached.

At the operation of the trapping Filter 21 with such composition, the proximal and distal bundles is allowed to slide along the Shaft 13 and then the filter 21 is surely expanding and folding to the diameter direction, easily. Consequently, trapping filter 21 and the tip 14 may incline within a tubular cavity and would not damage a blood vessel wall.

Additionally, the trapping filter in the FIGS. 1 and 2 might be considered to be non-filter part with bundled strings of 1-20 wires as well as the trapping filter 21 in the 2nd example from proximal bundle 5 of wire 3 to flip over position.

Moreover, at the trapping filter 1 in the FIGS. 1 and 2, the proximal bundle part 32 and the distal bundle part 33 might be fixed to be definite slide distance or movability. Additionally, the Stopper ring 38 might be installed.

### INDUSTRIAL APPLICATION

This invention has the industrial availability to trap the thrombus perfectly in blood vessel with assured expansion of trapping filter from metallic wire by visualization from extra-corporeal at the filter of parasol or parachute shape.

### TECHNICAL APPLICATION

This invention is the trapping filter and related trapping system. On details, this relates the trapping filter for plaque or thrombus in the blood and related trapping system, which expands in the tubular cavity in the body.

What is claimed is:

1. An expandable trapping filter for a blood vessel operative on a guide wire shaft disposed therein comprising:
a plurality of knitted metallic wires having a memory shape characteristic;
said knitted metallic wires forming a filter having two bundled parts, one bundled part at a distal filter end and the other bundled part being at a proximal filter end of plurality of knitted wires;
wherein, when said filter expands in said blood vessel, each wire flips over to form parasol shape moving proximal bundled knitted wires to distal bundled knitted wires, said distal bundled knitted wires forming a mesh-like filter to catch a thrombus therein.

2. The filter as claimed in claim 1 comprising plural knitted metallic wires of up to 12 wires.

3. The filter as claimed in claim 1 comprising a bent-over portion of one to 12 wires in the proximal bundled portion.

4. The filter as claimed in claim 1 wherein said knitted wire is more than 20 wires.

5. The filter as claimed in claim 1 wherein said wire is shape memory wire, selected from metallic alloys of Nickel-Titanium, Copper-Zinc-Aluminum, Copper-Aluminum-Nickel or Iron-Manganese-Silicon and/or radiopaque metal.

6. The filter as claimed in claim 5 wherein said trapping filter is composed by the radiopaque material disposed on or in the core of the wire.

7. The filter as claimed in claim 6 wherein said radiopaque metal is gold or platinum with core diameter is 10-80% of wire outside diameter.

8. The filter as claimed in claim 1 wherein said filter is loaded in an interior of a sheath and said filter longitudinally moves in and out from sheath by relative movement of shaft and sheath.

9. The filter as claimed in claim 8 wherein said shaft is made from metallic material and has tapered fine shape at its distal end.

10. The filter as claimed in claim 1 wherein said trapping filter maintains the moving allowance up to define distance by shifting the distal or proximal bundled portion along the shaft direction.

11. The filter as claimed in claim 10 wherein said trapping filter has stopper to regulate the moving distance of distal or proximal bundled portion on the shaft.

12. The filter as claimed in claim 8 wherein the trapping filter has the guide tip of coil or ribbon shape from the distal bundled portion.

13. The filter as claimed in claim 8 wherein the trapping filter is composed of partial radiopaque metal on tip or shaft.
